(19) United States
(12) Patent Application Publication
(10) Pub. No.: US 2014/0221892 A1
(43) Pub. Date: Aug. 7, 2014

(54) MATERNITY SUPPORT BELT

(71) Applicant: MAMA DOC, LLC, (US)

(72) Inventors: Sumbal Javaid, Mason, OH (US); Kimberly Howell, Mason, OH (US)

(73) Assignee: MAMA DOC, LLC, Mason, OH (US)

(21) Appl. No.: 13/757,519

(22) Filed: Feb. 1, 2013

Related U.S. Application Data
(60) Provisional application No. 61/594,908, filed on Feb. 3, 2012.

(51) Int. Cl. A61F 5/02

(52) U.S. Cl. CPC A61F 5/028
USPC 602/19

(57) ABSTRACT

A maternity support belt includes a curved support edge matching the female pelvic curve to support a gravid abdomen. The support belt has an elongated shape and is sized and configured to wrap around the periphery of a pregnant female. The maternity belt includes a lumbosacral supporting section that applies a compressive force against the lumbosacralportion of the wearer's back, and a pair of gravid abdominal support sections on opposite lateral sides of the lumbosacral supporting section and contiguous thereto. The gravid abdominal support sections adjustably overlap and connect to each other on the wearer's front side. The maternity support belt is formed of a rayon from bamboo material, which is wicking and thermo-regulatory.

### RELATED APPLICATIONS

This application claims priority under 35 U.S.C. §119(e) to United States Provisional Application Ser. No. 61/594,908, filed Feb. 3, 2012.

### TECHNICAL FIELD AND INDUSTRIAL APPLICABILITY OF THE INVENTION

The invention relates generally to the field of maternity support belts and will be disclosed in connection with a pregnancy support belt that supports both the back of a pregnant wearer and includes a unique curve that follows the pelvic curve of the female form and supports a gravid abdomen.

### BACKGROUND OF THE INVENTION

It is estimated that approximately 68% of pregnant women experience back pain at some point during pregnancy. Forty-five percent of these women experience leg pain. The pain comes from a number of factors, including weight gain, the changing curvature of the spine and the growing uterus. These changes in a woman's body place strain on both nerves and muscles of the back and legs. Unfortunately, current therapies available to alleviate this pain are effective only about 25% of the time. Moreover, pharmacologic options to alleviate the pain and discomfort are highly limited due to potential risk that the drugs will be passed to the fetus, and have deleterious effects on the fetus. Therefore, although the problem is widespread nature of this problem, expectant mothers have few realistic options for relief, and are forced to suffer from the pain and discomfort throughout their pregnancy. Prior to this invention, many support belts were offered to women. However, these belts were cumbersome to use, and not fully effective in correcting the multiple problems cause by a gravid abdomen.

### SUMMARY OF THE INVENTION

In one example of the invention a maternity support belt includes an elongated elastic support member that is sized and configured to wrap around the bi-lateral lower quadrants of a body of a female wearer, over the hips and pubic bone and under a gravid abdomen. The elongated support member extends between first and a second lateral end portions. The support member has a lumbosacral supporting section and a pair of laterally outward extending gravid abdominal support sections on opposite lateral sides of the lumbosacral supporting section. The lumbosacral supporting section is configured and adapted to provide a compressive supporting force about a wearer's lumbar spine, and the gravid abdominal support sections is sized and configured to provide subjacent support to a wearer's gravid abdomen. The gravid abdominal support sections of the support member are adapted to wrap around the periphery of a wearer's body and to overlappingly connect to each other at a location peripherally spaced from the lumbosacral supporting section. The elongated support member has upper and lower edges that extend between the first end and second end portions. The upper edge extends from the first end portion to the second end portion along a curved path that generally corresponding to a female pelvic curve.

In another example of the invention, the maternity support belt includes a lower edge that extends between the first and second end portions in a generally straight line.

In another example of the invention, the maternity belt has upper and lower edges that are separated from each other in the lumbosacral supporting section by approximately 6 inches.

In another example of the invention, the upper and lower edges of the support are separated at the gravid abdominal support sections by approximately ⅔ of the separation distance between the upper and lower edges in the lumbosacral supporting section.

In another example of the invention, the upper and lower edges of the support are separated at the gravid abdominal support sections by approximately 3 to 4 inches.

In another example of the invention, the elongated dimensions of the fastening sections are approximately equal, and overlapping gravid abdominal support sections connect to each other, when worn by a wearer, at a location on the opposite side of a wearer from the lumbosacral supporting section.

In another example of the invention, the distance between the upper and lower edges is reduced by approximately 2.25 inches over longitudinal length of approximately 7⅛ inches.

In yet another example of the invention, he gravid abdominal support sections are each longitudinally contiguous to the lumbosacral supporting section.

In a still further example of the invention, the gravid abdominal support sections adjustably connect to each other.

In another example of the invention, the support belt is formed of non-latex material.

In another example of the invention, the support belt is formed of a wicking, thermo-regulatory material.

In another example of the invention, support belt is formed of a rayon material formed from bamboo cellulose.

In another example of the invention, adjustability of the gravid abdominal support sections is provided with hook and loop fasteners on the end portions of the support member, with one end including a hook component and the other end including a complementary loop component.

In another example of the invention, the maternity belt includes a pocket on the internal surface of the lumbosacral supporting section. The pocket is adapted to receive a thermal therapy device to transfer heat between the device and the lumbosacralarea of a wearer's back.

In one example of the invention, a maternity support allows a user to wear the support under her pregnant belly and on top of the pubic bone so as to support a pregnant wearer's hips, bladder, belly, fetus, and back.

In another example of the invention, a support belt is made of adjustable elastic, covered in rayon from bamboo.

In another example, the front closure of a support is configured to allow for approximately 10 inches of adjustability, which wide range of adjustability accommodates a typical woman for her entire period of gestation.

In a further example of the invention, a support belt is designed to fit firmly against the lumbar spine.

In another example of the invention, the belt fits over the hips and pubic bone and under the gravid abdomen. The design maximizes support of the abdomen, bladder, back, hips and baby during pregnancy.

### BRIEF DESCRIPTION OF THE DRAWINGS

While the invention concludes with claims which particularly point out and distinctly claim the invention, it is believed the present invention will be better understood from the following description taken in conjunction with the accompanying drawings, in which like reference numbers identify the same elements in which:

FIG. 1 is a side view of a pregnant woman wearing an exemplary form of a support belt constructed in accordance with the principles of the invention;

FIG. 2 depicts the support belt of FIG. 1 as it is disposed on a flat surface;

FIG. 3 illustrates the right end of the flattened support belt as viewed from the angle shown in FIG. 2 and depicts one component of a hook and loop fastening system disposed on the end sections;

FIG. 4 shows the backside of the support belt illustrated in FIG. 1 as it is worn by a user; and

FIG. 5 shows the inside inner pockete on the central portion of the support belt shown in FIG. 1 with a pocket designed for accepting a thermal conditioning pack, such as an ice or a heat pack.

Reference will now be made in detail to certain exemplary embodiments of the invention, examples of which are illustrated in the accompanying drawings.

### DETAILED DESCRIPTION OF THE PREFERRED EMBODIMENTS OF THE INVENTION

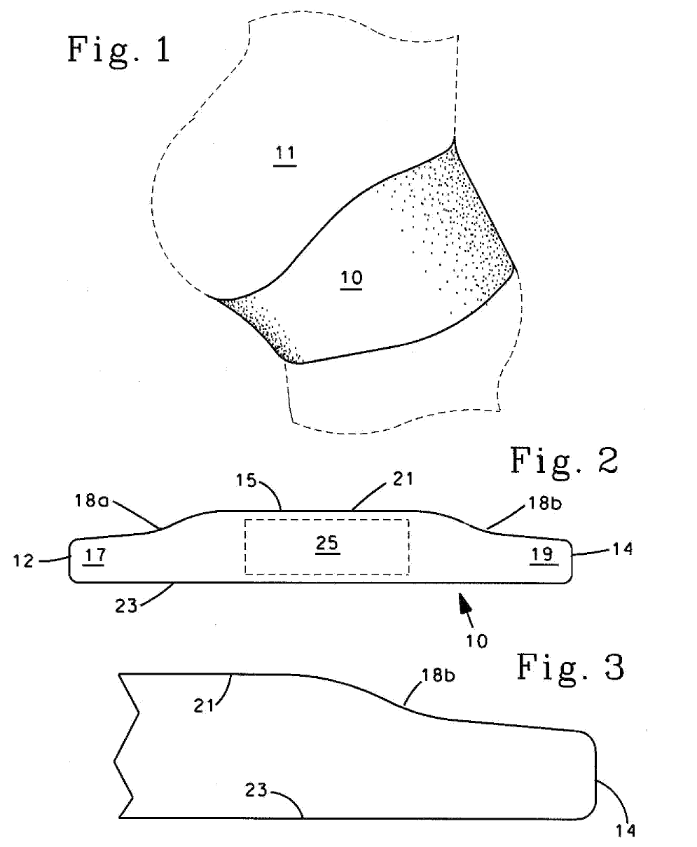

Referring now to the drawings, a maternity support belt constructed in accordance with the principles of the present invention, generally identified by the drawing numeral 10, is disclosed in the accompanying drawings. In general terms, the illustrated maternity support belt 10 is formed by an elongated elastic member with an elongated longitudinal expanse that extends from a first lateral end portion 12 to a second lateral end portion 14. (See FIG. 2). The elongated support belt 10 is sized and configured to wrap around the bilateral lower quadrants of a body of a female wearer (generally designated by the numeral 11, see FIG. 1), over the hips and pelvic bone and under a gravid abdomen, as shown in FIG. 1.

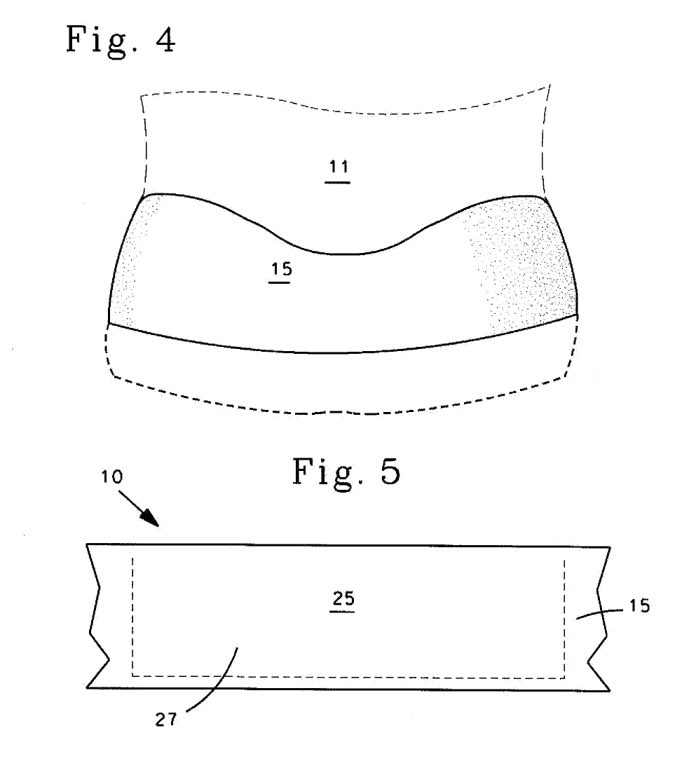

The maternity support belt 10 has a centrally disposed lumbosacral supporting section 15 and a pair of laterally outwardly extending gravid abdominal support sections 17 and 19. (See FIG. 2). The lumbosacral supporting section 15 is configured and adapted to provide a compressive supporting force about a wearer's lumbar spine (see FIG. 4), and the gravid abdominal support sections 17 and 19 are sized and configured to provide subjacent support to a wearer's gravid abdomen. (see FIG. 1 and 4). The gravid abdominal support sections 17 and 19 are adapted to wrap around the periphery of a wearer's body and to adjustability and overlappingly connect one of the gravid abdominal support sections 17 and 19 to the other at a location that is peripherally spaced from the lumbosacral supporting section 15, such as, for example, a location about the front of a wearer.

In the exemplary embodiment shown in the drawings, the gravid abdominal support sections 17 and 19 are laterally disposed and contiguous to the centrally disposed lumbosacral supporting section 15. With this configuration, the gravid abdominal support sections 17 and 19 overlap and interconnect with each other at a peripherally spaced location around the maternity support belt 10. When the gravid abdominal support sections 17 and 19 are of equal length, and equally spaced from the longitudinal center of the lumbosacral supporting section 15, the gravid abdominal support sections 17 and 19 overlap and connect at a location in the front of a wearer, on the opposite side of the wearer from the lumbosacral supporting section 15. In this configuration of the maternity support belt 10, the gravid abdominal support sections 17 and 19 overlap and interconnect at a position that makes it easy and convenient for the wearer to connect and disconnect the gravid abdominal support sections 17 and 19 from each other. While this configuration may be optimal in many circumstances, it will be appreciated that other configurations might be adopted wherein the gravid abdominal support sections 17 and 19 are of unequal lateral length, and/or overlap and connect at locations around the periphery of the support belt that are not directly in the front, center position of a wearer. This alternative location might be employed, and might be advantageous in some circumstances.

The upper and lower boundaries of the elongated support number 10 are defined by upper and lower edges 21 and 23, which edges 21 and 23 extend between the first and second portions 12 and 14. In the exemplary embodiment specifically illustrated in the drawings, the lower edge 23 is generally a straight line. The top edge 21, however, extends between the end portion 12 and 14 along a curved path. Significantly, the top edge 21 has a shape that generally corresponds to the shape of the female pelvic curve. This curved path is created by the differing heights of the lumbosacral supporting section 15 and the gravid abdominal support sections 17 and 19. More specifically, as best depicted in FIGS. 2 and 3, in the gravid abdominal sections, the upper edge 21 is separated from the lower edge 23 by approximately two thirds of the separation distance between the upper and lower edges 21 and 23 in the lumbosacral supporting section 15. In the particular exemplary embodiment depicted in the drawings, the upper edge 21 is separated from the lower edge 23 by approximately 3¾ inches from the first end 12 to the curve 18a, and then is separated from the lower edge 16 by about 6 inches from the curve 18a to the curve 18b. From the curve 18b to the second end 14, the upper edge 21 is separated from the lower edge 23 by about 3¾ inches. The curves 18a and 18b are gradual curves wherein that extend over a length of approximately 7⅛ inches. As noted above, the curve of the upper edge 21 designed to follow the pelvic curve of the female form to allowing the user to wear the support belt under the pregnant belly and on top of the pubic bone. This configuration allows maximal support of the pregnant hips, bladder, belly, baby and back. The curve, best shown in FIGS. 2 and 3 thus falls 2.25 inches from the maximum height of the device.

The elongated elastic support member 10 is preferably completely adjustable over a wide range of dimensions, approximately 10 inches in the exemplary embodiment depicted in the drawings. This adjustability is achieved through use of hook and loop fasteners (of the type commonly sold under the trademark Velcro) on the longitudinal ends of the gravid abdominal support sections 17 and 19. Specifically, one of the ends 12 or 14 includes a hook component of a hook and loop testing system, and the other includes a complementary loop component.

The elongated maternity belt 10 preferably is made of non-latex elastic and is covered with a rayon from bamboo material, which is wicking and thermo-regulatory.

The side of the belt which sits against the patients back has a built in pocket 25 for a thermal therapy packet, such as an ice/heat pack 27 as shown in FIG. 5. In one exemplary embodiment, the pocket 25 has dimensions of approximately 10.5 inches by 4.25 inches and accommodates various cooling/heating devices. This allows a pregnant woman to safely delivery heat/cooling to sore lower back muscles while at a safe distance from the fetus.

FIG. 4 shows the back of the portion of the maternity support belt 10, and specifically the lumbosacral supporting section 15, as it is worn across the lumbar spine. As those familiar with pregnancy will appreciate, a pregnant woman experiences an exaggerated lordosis in her back, and lordosis places the back muscles under substantially increased tension due to the increased curvature of the spine and the enlarged abdomen and uterus. Heat therapy from the thermal therapy pack 27 increases circulation and relaxes the back muscles, alleviating much of the pain and discomfort typically experienced by pregnant women.

FIG. 3 illustrates the front closure device, which can be engaged under the gravid abdomen. This hook and loop closure allows substantial adjustability, approximately 10.5 inches of adjustability in one exemplary embodiment. This allows the wearer the flexibility of using the maternity belt 10 for the purpose of supporting her belly, back, hip and bladder support for all 3 trimesters of pregnancy. As will be appreciated from the above, one of the ends 12 or 14 includes a hook component of a hook and loop fastening system, and the other end includes the complementary loop component.

The foregoing description of the preferred embodiments of the present invention have been presented for purposes of illustration and description. It is not intended to be exhaustive or to limit the invention to the precise form disclosed. Obvious modifications or variations are possible in light of the above teachings. For example, . . . The embodiments were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally and equitably entitled. The drawings and preferred embodiments do not and are not intended to limit the ordinary meaning of the claims in their fair and broad interpretation in any way.

What is claimed is:

1. A maternity support belt, comprising:
an elongated elastic support member sized and configured to wrap around the bi-lateral lower quadrants of a body of a female wearer, over the hips and pubic bone and under a gravid abdomen;
the elongated support member extending between first and a second lateral end portions;
the support member having a lumbosacral supporting section and a pair of laterally outward extending gravid abdominal support sections on opposite lateral sides of the lumbosacral supporting section, the lumbosacral supporting section being configured and adapted to provide a compressive supporting force about a wearer's lumbar spine, and the gravid abdominal support sections being sized and configured to provide subjacent support to a wearer's gravid abdomen;
the gravid abdominal support sections of the support member being adapted to wrap around the periphery of a wearer's body and to overlapping connect to each other at a location peripherally spaced from the lumbosacral supporting section;
the elongated support member having upper and lower edges that extend between the first end and second end portions, the upper edge extending from the first end portion to the second end portion along a curved path that generally corresponding to a female pelvic curve.

2. A maternity support belt as recited in claim 1 wherein the lower edge extends between the first and second end portions in a generally straight line.

3. A maternity support belt as recited in claim 1 wherein the upper and lower edges of the support are separated by approximately 6 inches.

4. A maternity support belt as recited in claim 1 wherein the upper and lower edges of the support are separated at the gravid abdominal support sections by approximately ⅔ of the separation distance between the upper and lower edges in the lumbosacral supporting section.

5. A maternity support belt as recited in claim 3 wherein the upper and lower edges of the support are separated at the gravid abdominal support sections by approximately 3 to 4 inches.

6. A maternity support belt as recited in claim 1 wherein the elongated dimensions of the fastening sections are approximately equal, and overlapping gravid abdominal support sections connect to each other, when worn by a wearer, at a location on the opposite side of a wearer from the lumbosacral supporting section.

7. A maternity support belt as recited in claim 3 wherein the distance between the upper and lower edges is reduced by approximately 2.25 inches over longitudinal length of approximately 7⅛ inches.

8. A maternity support belt as recited in claim 1 wherein the gravid abdominal support sections are each longitudinally contiguous to the lumbosacral supporting section.

9. A maternity support belt as recited in claim 1 wherein the gravid abdominal support sections adjustably connect to each other.

10. A maternity support belt as recited in claim 9 where the support member is formed of non-latex material.

11. A maternity support belt as recited in claim 1 wherein the support member is formed of a wicking, thermo-regulatory material.

12. A maternity support belt as recited in claim 1 wherein the support member is formed of a rayon material formed from bamboo cellulose.

13. A maternity support belt as recited in claim 9 wherein the adjustability of the gravid abdominal support sections is provided with hook and loop fasteners on the end portions of the support member, with one end including a hook component and the other end including a complementary loop component.

14. A maternity support belt as recited in claim 1 further including a pocket on the internal surface of the lumbosacral supporting section, the pocket being adapted to receive a thermal therapy device to transfer heat between the device and the lumbosacralarea of a wearer's back.
